(19) United States
(12) Patent Application Publication
(10) Pub. No.: US 2008/0032375 A1
(43) Pub. Date: Feb. 7, 2008

(54) Method and Fermenter for the Anaerobic Fermentation of Biological Waste

(76) Inventors: Rudolf Hartmann, Gelterkinden (CH); Hans Wuethrich, Kienberg (CH)

Correspondence Address: OLIFF & BERRIDGE, PLC, P.O. BOX 320850, ALEXANDRIA, VA, 22320-4850, US

(21) Appl. No.: 11/596,811

(22) PCT Filed: May 19, 2005
(86) PCT No.: PCT/EP05/05452

§ 371 Date: Jan. 30, 2007

(30) Foreign Application Priority Data
May 19, 2004 (DE) 10 2004 025 318.8

(51) Int. Cl. C12P 1/04; C12M 1/02; C12M 1/04; C12M 1/107

(52) U.S. Cl. 435/170; 435/296.1; 435/298.1; 435/300.1

(57) ABSTRACT

The invention relates to a method for the anaerobic fermentation of biological waste and a fermenter for carrying out said method. According to the invention, the starting material, in other words, the biological waste for treatment, is introduced through several inlet openings distributed along the reactor height or length and/or fermented product is extracted through several fermented product outlet openings.

The invention relates to a method of anaerobic fermentation of biological waste in accordance with the preamble of claim 1 and a fermenter, especially for carrying out said method.

Upon introduction of the separate collection of organic household refuse in Europe, the mechanically biological recovery (German abbreviation: MBA) of urban refuse has become increasingly important. The decomposition of the biogenic mass takes place on a microbial basis, wherein a difference can be made between aerobic and anaerobic microorganisms. The aerobic reaction ultimately results in the final products carbon dioxide and water and is referred to as rotting. The anaerobic reaction is typical of fermentation; the final products formed are, inter alia, methane, ammonia and hydrogen sulfide.

In DE 196 48 731 A1 an aerobic method is described in which the organic components of a waste fraction are washed out in a percolator and the residue is burnt or deposited, for instance, after drying.

The percolation can be carried out, for example, in a box percolator according to WO 97/27158 A1. Also tests using a boiling percolator according to DE 101 42 906 A1 in which the percolation is carried out in the boiling range of the process water turned out to be promising.

The organically highly loaded exit water extracted from the percolator is supplied to a biogas plant for anaerobic decomposition, wherein the organic part is converted by means of methane bacteria and can be fed to biogas combustion via a gas-making pipeline for energy generation. The afore-described aerobic treatment of the waste materials in a percolator has turned out to be extremely competitive with the anaerobic methods and has become increasingly important.

In EP 0 192 900 B1 the Valorga method, as it is called, is described—in which the fermentation is carried out in a fermenter which is charged from the bottom. The waste to be recovered is guided in plug shape to an outlet arranged below the radially outer inlet opening. The waste is conveyed by blowing in compressed biogas via gas nozzles disposed in several sectors of the fermenter, wherein each sector can be individually controlled to maintain the plug

In a particularly preferred embodiment the impurity/high-gravity solids are conveyed and discharged to the center of the fermenting reactor via two conveying means.

The introduction and the extraction of starting material/fermented product is preferably carried out via a central conveying station by which the flow paths can be reversed to and from the inlet/outlet openings and thus appropriately varying material flow profiles can be formed in the fermenting reactor.

The formation of such material flow profile is supported by an agitator the direction of rotation of which can be reversed during the fermenting process.

In an advantageous embodiment of the invention, neighboring mixing blades of the agitator overlap in axial direction so that a complete mixing of the reactor content is ensured.

The agitator may have an especially simple design, when the agitating shaft thereof is supported on both sides in the reactor and the diameter is dimensioned such that the agitating shaft is sufficiently supported by the buoyancy occurring in the reactor.

The fermenting reactor is preferably horizontally arranged and has a circular or approximately trapezoidal cross-section. In the latter case two inclined surfaces and one horizontal surface disposed therebetween are formed in the area of the reactor bottom.

The gas injection nozzles for injecting biogas are disposed in the area of the two inclined surfaces in a reactor having a trapezoidal cross-section.

The gas injection nozzles can open in vertical direction, i.e. in parallel to the vertical reactor axis or normal to the inclined surfaces.

For adjusting an optimum operating temperature the shell of the fermenter can be heated.

In the event that the material flows are controlled by a central conveying station, in addition a separate direct feeding of starting material can be provided through which starting material can be fed independently of the conveying station.

The assembly of the fermentation plant according to the invention is especially simple, when the fermenting reactor is composed of segments ready for transport which then are assembled on the spot at the construction site.

Other advantageous further developments of the invention are the subject matter of further subclaims.

Hereinafter preferred embodiments of the invention are explained in detail by way of schematic drawings, in which

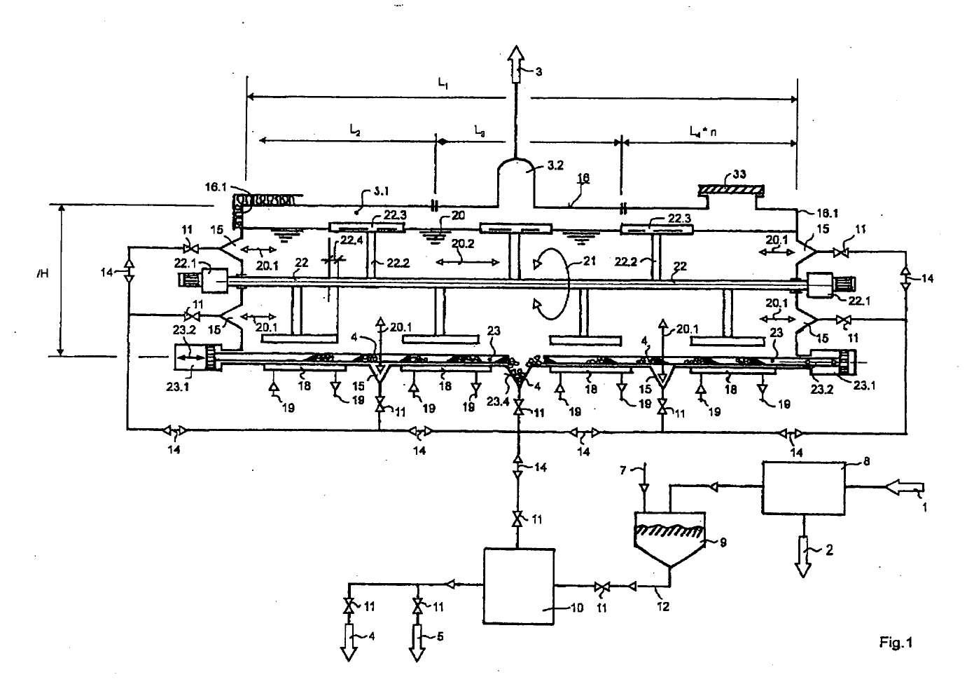

FIG. 1 shows a process diagram of the process according to the invention for anaerobic fermentation of biological waste comprising a fermenting reactor according to the invention;

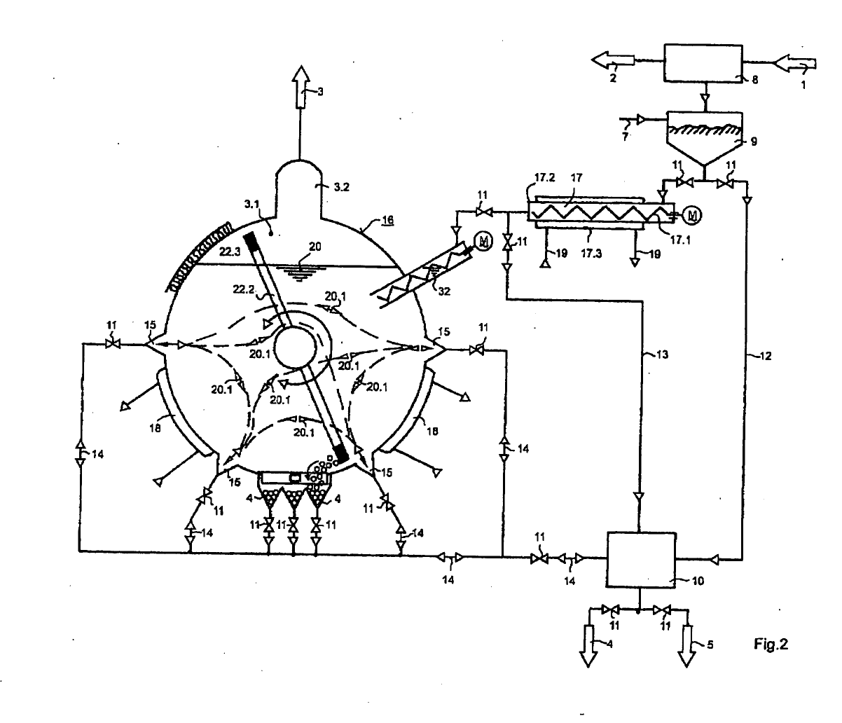

FIG. 2 shows a side view of the fermenting reactor of FIG. 1;

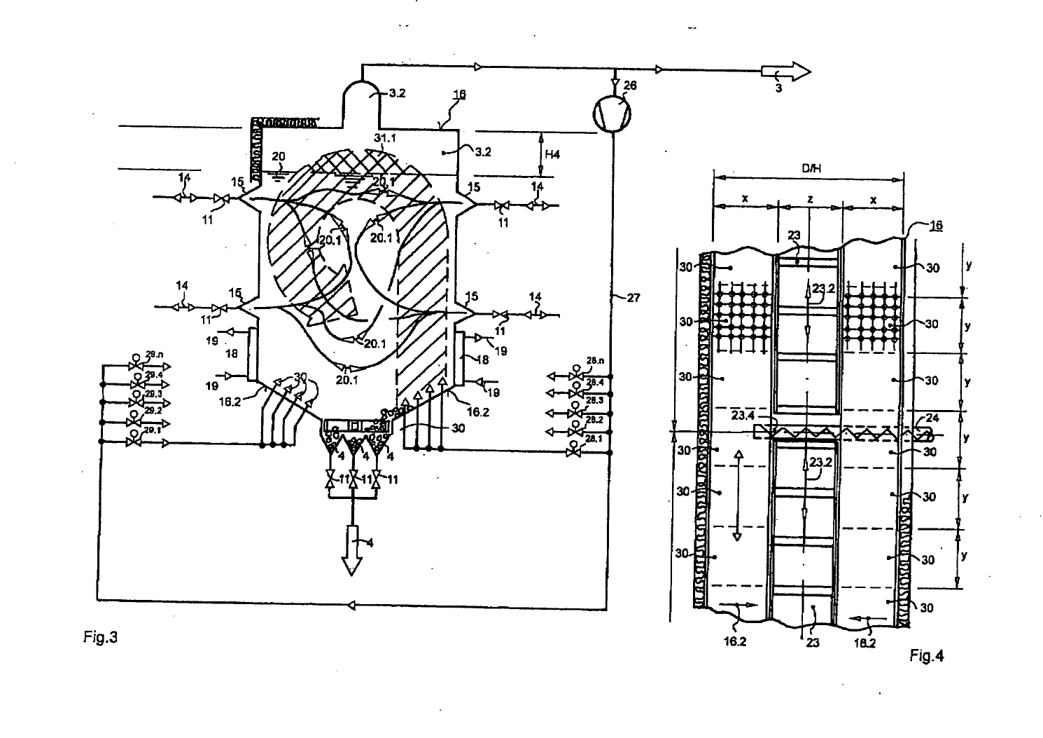

FIG. 3 shows a side view of another embodiment of a fermenting reactor and

FIG. 4 is a cut top view of the fermenting reactor of FIG. 3;

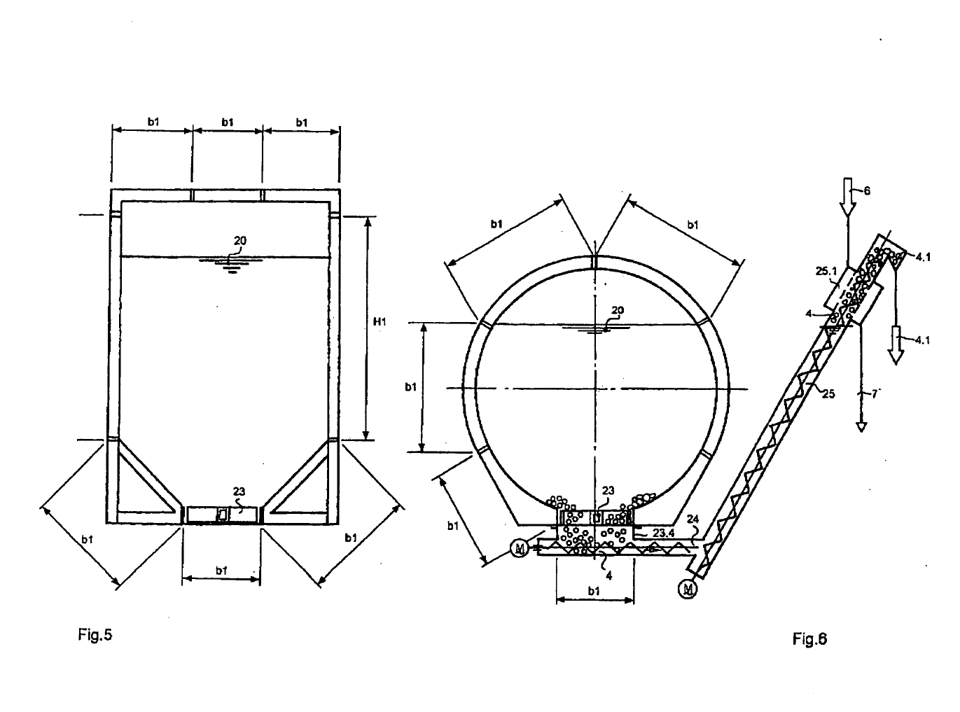

FIG. 5 shows the fermenting reactor of FIG. 3 in segmental design and

FIG. 6 shows the fermenting reactor of FIG. 2 in segmental design and comprising a high-gravity solids extracting system.

In FIG. 1 the process diagram of a process according to the invention for anaerobic fermentation of biogenic waste is shown. The introduced starting material 1 contains domestic waste (residual waste), for instance, having a comparatively high organic component, biological waste from the separate collection, organically highly loaded waste from food industry and excessively stored food, slaughtering waste, organically enriched slurry such as e.g. active slurry from sewage plants. From this starting material 1 impurities 2 as well as impurity/high-gravity solids 4 occurring in process steps hereinafter described in detail are eliminated and the remaining starting material 1 is supplied to a fermenting reactor 16. In the latter fermenting gases are formed as metabolic product from the fermenting process, especially biogas 3 (methane gas) which is extracted to the top. The fermented product largely freed from the organic components is extracted after completion of the fermenting process and is supplied to further treatment, such as e.g. dehydration, drying or composting. According to legal provisions, fermented product from residual waste must be deposited or burnt or at least recovered into substitute fuels. Fermented product from biological waste or renewable raw materials can be used as fertilizer or soil conditioner after dehydration and further composting.

According to FIG. 1, the entering starting material 1 is thus decomposed into impurity/high-gravity solids 2, 4, fermented product 5 and biogas 3.

The starting material 1 fed is initially supplied to a mechanical accepting and preparation plant 8 in which the impurity solids 2 are sorted, crashed and extracted. Moreover, in this accepting and preparation plant 8 excessively stored food is unpacked and loading material and liquid waste, by which the dry matter content is adjusted, are added and conditioned.

The prepared and conditioned starting material is then fed to a pump collecting tank 9 and there possibly mixed with sewage 7 occurring during purification of high-gravity solids according to FIG. 6, as will be described further below.

The collecting tank 9 is connected via a pipeline 12 and slides 11 to a central pump/conveying station 10 by which practically all substantial material flows of the plant are controlled.

The pump/conveying station 10 can be operated both in suction and in pressure operation so that either starting material 1 is conveyed from the collecting tank 9 via pipelines 14 and appropriately adjusted slides 11 to inlet openings 15 or fermented product 5 can be extracted via the pipelines 14 and appropriately reversed slides 11 as well as impurity/high-gravity solids can be extracted via a central extract opening 16.3 from the fermenting reactor 16.

According to FIGS. 1 and 2, the fermenting reactor 16 has an approximately cylindrical structure and is horizontally disposed, wherein along its outer diameter and its length a plurality of inlet and outlet openings 15 and the central extract opening 16.3 are provided. The inlet/outlet openings 15 can be used, depending on the control via the central pump/conveying station 10 and on the appropriate adjustment of the slides 11, as inlet opening for starting material or outlet opening for fermented product. As indicated in broken lines in FIG. 2, by this adequate control a desired material flow between the inlet/outlet openings 15 can be adjusted which is selected such that an optimum mixing of the fermented product is ensured. Moreover, the pump/conveying station 10 permits to extract fermented product via one of the inlet/outlet openings 15, for instance, and then to re-feed it as inoculum via a different one of the inlet/outlet openings 15. The guiding of the flow, for example, is chosen such that inside the reactor no substantial differences in concentration of organic acids and of ammonium are adjusted so that the fermenting process can take place in the predetermined manner.

In the pump/conveying station 10 preferably rotary piston, displacement or suction/pressure tank systems are employed as conveying means, which are used, for instance, in agriculture or for sewerage clearance. By appropriate adjustment of the slides 11 then the following functions can be carried out basically by the pump/conveying station 10:
a) Suction of starting material 1 from the collecting tank 9 via the pipeline 12;
b) Introduction of starting material 1 from the collecting tank 9 through the inlet and outlet openings 15 into the reactor 16 or
c) Circulation of the reactor content or fermenting sludge 20 in different places of the reactor 16 and in different directions through the inlet and outlet openings 15 as well as appropriate slide positions 11 and through the pipelines 14.

Further functions will be illustrated hereinafter by way of FIG. 2.

The cylindrical, horizontally disposed fermenting reactor 16 shown in FIGS. 1 and 2 comprises an agitator 22 driven by two torque-based gear motors 22.1 mounted on the face of the reactor 16. Said motors are controlled via frequency converters and thus their direction of rotation can be reversed periodically and/or in response to other operating parameters. Agitating arms 22.2 evenly distributed along the circumference or disposed in a plane are fastened to an agitator shaft 22.4 and extend in radial direction outwardly toward the circumferential wall of the fermenting reactor. Agitator blades 22.3 extending in parallel to the axis are fastened to the radially outer end portions of the agitator arms 22.2, wherein the radial length of the agitator arms 22.2 is selected such that the agitator blades 22.3 skim over the fermenting sludge level 20.1 during rotation so that a forming scum layer is destroyed or at least mixed.

In large plants the axial length of the fermenting reactor 16 may easily be more than 30 meters. As, according to the invention, it is endeavored to provide as few internal parts as possible inside the fermenting reactor 16, an agitator shaft 22.4 is dimensioned so that it is supported by the buoyancy of the fermenting sludge 20 in the fermenting reactor 16 and thus cannot sag—hence an expensive mounting inside the reactor chamber can be dispensed with.

Above the fermenting sludge level 20.1 a gas chamber 3.1 opening into a gas dome 3.2, from which the biogas 3 is extracted, is formed in the fermenting reactor 16. At the reactor bottom 2 two settled material discharge means are provided which are in the form of two interacting pusher plates 23 in the embodiment shown in FIG. 1. The latter convey the settled material in axial direction to the centrally disposed extract opening 16.3 through which the settled material (high-gravity/impurity solids) can be

This object is achieved, regarding the method, by the combination of features of claim 1 and, regarding the fermenter, by the combination of features of claim 10.

According to the invention, the anaerobic fermenting reactor (fermenter) is provided with plural inlet openings and fermented product outlet openings by which starting material or fermented product (the latter as inoculum) can be supplied and/or fermented product can be extracted. By this plurality of inlet and outlet openings the metabolic process can be controlled so that the concentration of organic acids and ammonium inside the fermenting reactor can be most largely evened. In the conventional plug-flow solutions described in the beginning, in the different longitudinal sections of the reactor different concentrations are brought about which considerably inhibit or even bring the fermenting process to a standstill and thus considerably extend the holding time.

In prior art this is supported by the fact that the inoculation with active bacteria mass or possibly a dilution with pressurized water is carried out exclusively in the area of the inlet openings and during the entire holding time channeling and thus short-circuit flows between the inlet and the outlet side are avoided.

In the solution according to the invention making use of starting material/inoculum supply through several inlet openings and, where appropriate, also extraction of fermented product through plural outlet openings, the fermented product is partly mixed and inoculum is introduced along the flow path of the waste to be treated inside the reactor—this results in the fact that the holding time can be reduced to a fraction of the holding times required in prior art. It is expected that the holding time in the solution according to the invention is less than two days.

In an especially preferred embodiment the fermented product is thoroughly mixed inside the fermenting reactor via a mechanical agitator and/or by biogas injection so that the fermenting process is further improved.

It is especially preferred in this context that the direction of rotation of the agitator is reversed during the fermenting process so as to further improve thorough mixing.

The biogas is preferably injected into the fermenting reactor by gas injection nozzles disposed in the reactor bottom. The gas injection nozzles are preferably combined in fields and are successively controlled. The gas injection is controlled such that the scum is broken up in the area of the respectively controlled field. flow of the waste between the inlet opening and the outlet opening.

In EP 0 476 217 A1 a heatable fermenter is disclosed in which starting material and sludge material are supplied to the fermenter as bacteria inoculum and the sludge material formed is transported to a sludge material outlet via an agitator. Such an addition of inoculum may also be provided in the Valorga method according to EP 0 192 900 B1 described in the beginning.

In DE 196 24 268 A1 a fermenting method for waste in fluid form, i.e. having a dry matter content (German abbreviation: TS) of less than 25%, is disclosed. A multi-chamber reactor is used for this purpose, wherein the fermented product can be transported from an inlet opening through the chambers to an outlet opening via an agitator. A common gas chamber from which the biogas formed during the fermenting process is extracted is allocated to the multi-chamber reactor. The metabolism can be individually controlled in the individual chambers by a different conduct of the process, for instance via heat exchangers, addition of inoculum etc.

EP 0 794 247 A1 discloses a fermenter in which the fermented product is introduced to a rotating drum in which a spiral is arranged. The fermented product is guided in plug shape from the inlet to the sludge material outlet via said spiral. This supply can take place by forward and backward rotation of the drum, wherein the forward rotation, i.e. the transportation of the fermented product in the direction of the fermented product outlet takes longer than in the opposite direction so that a predetermined holding time of the fermented product is reached.

Since the waste to be treated also contains a quite considerable part of high-gravity solids and impurities, especially the solutions using mechanical conveying means (EP 0 794 247 A1, EP 0 476 217 A1, DE 196 24 268 A1 ) are subjected to relatively high wear, because the conveying means employed and other internal parts can be damaged by the sediments including the impurity/high-gravity solids.

Moreover, all fermenting processes require very long holding times of from 18 to 30 days. Such holding times in turn require a considerable buffer capacity.

Compared to this, the object underlying the invention is to provide a method of anaerobic fermentation of biological waste as well as a fermenter by which the holding time can be substantially reduced vis-à-vis conventional solutions. discharged. The two pusher plates 23 are driven by a cylinder/piston unit 23.1 adapted to be operated electrically or hydraulically. By said cylinder/piston unit 23.1 the pusher plates 23 perform strokes in the directions of the arrows 23.2 so as to convey the settled material in the direction of the extract opening 16.3. In the view according to FIG. 1, the agitator blades 22.3 end somewhat above the pusher plates 23 so that the settled material is conveyed downward inside the reactor by the agitator 22. The gas chamber 3.1 is secured by a safety means 33 so that no excessive pressure can build up.

The above-mentioned control of the gear motors 22.1 of the agitator 22 is designed such that the settled material 4 is introduced evenly from both sides into a discharge shaft of the pusher plates 23 by reversing the direction of rotation and appropriate timing.

According to FIGS. 1 and 2, a shell 16.1 of the fermenting reactor 16 is provided with insulation 16.1 to maintain a predetermined fermenting temperature. This fermenting temperature can be adjusted by means of heating pockets 18 (FIG. 2) distributed along the outer circumference of the fermenting reactor 16 and can be controlled by the plant control in such way that inside the reactor the predetermined temperature profile is adjusted.

As one can take from FIG. 2, in addition to the material flows (starting material, fermented product, inoculum) adjusted by the central pump and conveying station 10 which can be introduced or extracted via the inlet and outlet openings 15, starting material can be further introduced via direct charging. Said starting material is branched off by an appropriately adjusted slide 11 and heated to processing temperature by a heat exchanger 17. The heat exchanger 17 is surrounded by a heating shell 17.3 and includes a guiding tube 17.2 heated thereby in which a conveyor spiral 17.1 is disposed through which the starting material is introduced and further conveyed. The starting material 1 heated to processing temperature is then conveyed into the interior of the reactor via a further slide 11 and a spiral conveyor 32, for instance, wherein the spiral conveyor 32 enters below the fermenting sludge level 20.1.

Preheated starting material can be branched off downstream of the heat exchanger 17 via a further slide 11 and can be guided to the central pump/conveying station 10 via a branch line 13. It can be taken from the representation according to FIG. 2 that the extract opening 16.3 can be formed by three or more parallel extract areas 16.3a, 16.3b, 16.3c through which the settled material conveyed by the pusher plates 23 can be extracted toward the conveying pipelines 14 by way of slides 11a, 11b, 11c.

In FIG. 2 it is also illustrated very clearly that the agitator blades 22.3 shovel the settled material to the pusher plates 23 and, depending on the control of the slides 11, via the pump/conveying station 10 inside the fermenting reactor 16 different flow directions 20.2 of fermenting sludge are adjustable which result in an intense mixing and evening of the concentration inside the fermenting reactor 16.

The afore-described cylindrical reactor shape can be manufactured in a comparatively simple manner and is superior to other solutions as regards the compressive strength. Under certain conditions it can also be necessary, however, to design the fermenting reactor 16 to have a different geometry. Such embodiment is illustrated in FIGS. 3 and 4.

Accordingly, the fermenting reactor 16 has an approximately rectangular cross-section, the bottom being formed by two inclined surfaces 16.4 which are connected to each other by a horizontally extending horizontal surface 16.5. In the area of said horizontal surface 16.5 the two pusher plates 23 and the extract opening 16.3a, b, c are formed.

The inlet and outlet openings 15 are then provided in the side walls of the fermenting reactor 16 extending in vertical direction.

The material flows are controlled—as in the afore-described embodiment—by the central pump/conveying station 10 so that inside the fermenting reactor 16 in turn different material flow paths 20.2 can be adjusted.

In contrast to the afore-described embodiment, according to the solution shown in FIGS. 3 and 4 a gas injection plant is used instead of a mechanical agitator 22, i.e. a pneumatic agitation is used.

The gas injection plant has a plurality of nozzles 30.1 which preferably open in the inclined surfaces 16.4 of the fermenting reactor 16. In FIG. 3 two different nozzle orifice areas are shown. In the left-hand part of FIG. 3 the nozzles 30.1 extend approximately normal to the inclined surface 16.4, while the nozzles 30.1 in the right-hand part are arranged in parallel to the normal axis (vertical in FIG. 3) of the fermenting reactor 16. I.e. in the case of arrangement of the nozzles 30.1 as shown in FIG. 3 on the left, the injected gas flows into the reactor chamber obliquely with respect to the normal axis, whereas in the embodiment shown on the right it is injected in parallel to the normal axis.

For a pneumatic conveying and circulation of the fermenting sludge 20 biogas is used which is sucked from the gas dome 3.2 by means of a compressor 26 and then is guided via a gas injecting line 27 as well as via plural control valves 28, 29 and connected branch lines to a respective nozzle field 30 consisting of a plurality of nozzles 30.1.

As can be taken especially from the top view in FIG. 4, the fields 30 are arranged successively along the inclined surfaces 16.4 in the longitudinal direction of the reactor (normal to the plane of projection in FIG. 3), wherein biogas can be separately applied to each field 30 by the system control. The compressor 26 is arranged above the fermenting sludge level 20.1 by the measure H4 so that in the case of standstill of the compressor 26 no fermenting sludge 20 can penetrate the compressor via the gas injecting line 27.

The minimum gas pressure required for circulating the fermenting sludge 20 approximately corresponds to the barometric height (H2×1.5 (bar)) of the filling level required to overcome the pipeline resistance. The number of gas injection nozzles 30.1 per nozzle field 30 also depends on the dimensions x, y, i.e. the length and the width of the nozzle fields 30, wherein between 8 and 16 nozzles are disposed per square meter bottom area depending on the height H2.

The fields 30 are successively subjected to pressurized gas in longitudinal direction by alternately switching the control valves 28, 29. The fermenting sludge 20 is displaced by the ascending gas bubble and is moved by the occurring suction in the direction of the arrow according to FIG. 3, wherein the nozzles 30.1 opening in vertical direction initially bring about an upwardly directed flow, while the obliquely opening nozzles 30.1 deflect the fermenting sludge flow to the right.

The circulation can also take place inversely to the direction of the arrow by an appropriate control of the pump/conveying station 10 and the gas injection nozzles 30.1.

The time of applying gas via the nozzles 30.1 depends on the height of the tank H2, H3 and the adjusted dry matter content (TS). Gas is applied to each field 30 until a forming scum 31.1 is torn.

By the adjusting guiding of flow shown in FIG. 3 the settled material deposits at the inclined surfaces 16.4 and, due to the gradient, is conveyed toward the two pusher plates 23 by which the settled material is conveyed to the centrally arranged extract openings 16.3.

The other guiding of flow corresponds to that of the embodiment from FIG. 1 so that further explanations can be dispensed with.

As already mentioned, the fermenting reactor 16 according to the invention can have a considerable length (30 m). Therefore it is not possible to transport the finished reactor vessel to the construction site. So far it has had to be manufactured on the spot, i.e. at the construction site so that considerable manufacturing expenditure is required. In accordance with the invention, it is provided to manufacture the fermenting reactor 16 of a plurality of elements ready for road transport which are then assembled on the site at comparatively low cost. For this purpose, the length L1of the vessel is divided into elements ready for transport having a length of about 12 to 15 m and a width b1 of about 3 to 4 m. In the case of a rectangular vessel according to the FIGS. 3 and 5, the building height H1 approximately corresponds to a transport length of about 15 m and a width B1 (corresponding to the width of the inclined surfaces 16.4 and the horizontal surface 16.5 in horizontal direction) of about 4 m.

In a circular reactor according to FIG. 6 the vessel is divided into a plurality of segments each having a width b1 of 3 to 4 m and the aforementioned length of about 12 to 15 m so that a comparatively easy transport to the construction site and a quick assembly on the spot are possible.

In FIG. 6 a high-gravity solids outlet means is shown. The high-gravity solids settled by the effect of the mechanical agitator 22 or by the pneumatic conveying through the nozzles 30.1 and conveyed from the pusher plates 23 to the centrally arranged extract openings 16.3 first get into a discharging spiral conveyor 24 feeding an inclined conveyor 25. By the latter the high-gravity solids 4 are conveyed obliquely upwards to a cleansing plant 25.1 provided above the fermenting sludge level 20.1. In said cleansing plant 25.1 the soiled high-gravity solids 4 are conveyed through a screening basket to which cleaning water 6 is applied from outside for rinsing out the soil so that cleaned high-gravity solids 4.1 are extracted. The soiled cleaning water 7 is returned to the collecting tank 9 (see FIGS. 1 and 2) and is used for adjusting the dry matter (TS) content there. The cleaned high-gravity solids 4.1 can be deposited or supplied to any other utilization. Industrial water or fresh water, for instance, can be used as cleaning water 6.

The fermented product 5 occurring in the foregoing processes is subjected to further treatment, for example dehydration, drying or composting.

The agitating movement (mechanical/pneumatic) is assisted by the afore-described guiding of the flow of the fermenting sludge inside the fermenting reactor 16 along the flow lines 20.2 in FIGS. 2 and 3, but primarily the inoculation of the introduced starting material with active bacteria mass (inoculum) from the outlet or in different positions at the reactor 16 is improved and thus the biological reaction is accelerated.

Of course, also a mechanical agitator can be added to the gas inlet nozzles according to FIG. 3. The gas injection nozzles can be used also in a fermenting reactor having a circular cross-section in accordance with FIG. 1.

A method for anaerobic fermentation of biological waste and a fermenter for carrying out said method are disclosed. According to the invention, the starting material, in other words, the biological waste for treatment, is introduced through several inlet openings distributed along the reactor height and/or length and/or fermented product is extracted through several fermented product outlet openings.

List of Reference Numerals

1 Starting material
2 impurity
3 biogas
3.1 gas chamber
3.2 gas dome
4 impurity/high-gravity solid
5 fermented product
6 cleaning water
7 sewage
8 preparation plant
9 collecting tank
10 pump-conveying station
11 slide
12 pipeline
13 branch line
14 conveying lines
15 inlet/outlet opening
16 reactor
16.1 reactor shell
16.2 insulation
16.3 extract opening
16.4 inclined surface
16.5 horizontal surface
18 heating pockets
20 fermenting sludge
20.1 fermenting sludge level
20.2 fermenting sludge flow direction
22 agitator
22.1 gear motor
22.2 agitator arm
22.3 agitator blade
22.4 agitator shaft
23 pusher plate
23.1 cylinder/piston unit
23.2 stroke
24 discharging spiral conveyor
25 inclined conveyor
25.1 cleaning plant
26 compressor
27 gas injection line
28 control valves
29 control valves
30 nozzle field
30.1 nozzles
31.1 scum
33 safety means

1. A method of anaerobic fermentation of biological waste having a dry matter (TS) content of <40%, which is introduced as starting material to a reactor in which biological components are converted to biogas and the fermented product is extracted through a fermented product outlet, wherein part of the fermented product can be returned to the reactor as inoculum,
wherein
the starting material and fermented product are selectively introduced through several inlet openings distributed along the reactor height and/or length and/or fermented product is extracted through several fermented product outlet openings.

2. A method according to claim 1, wherein also inoculum can be supplied through the inlet openings.

3. A method according to claim 1, wherein the fermented product is mixed by a mechanical agitator or by gas injection.

4. A method according to claim 3, first alternative, wherein the direction of rotation of the agitator is reversed during the fermenting process.

5. A method according to claim 3, second alternative, wherein biogas is injected through gas injection nozzles disposed in the reactor bottom.

6. A method according to claim 5, wherein gas injection nozzles are successively controlled field by field.

7. A method according to claim 6, wherein gas is injected through one field until a scum is broken up in this area.

8. A method according to claim 1, wherein settled material is extracted centrally from the bottom of the reactor.

9. A method according to claim 8, wherein at the bottom of the reactor two opposed flows of impurities/settled material are formed toward a central settled material/impurities outlet.

10. A fermenter, carrying out the method according to claim 1, comprising an anaerobic fermenting reactor to which starting material can be introduced and which has a gas dome for extracting biogas as well as a fermented product outlet for extracting fermented product, wherein a mixing means is provided in the reactor, wherein the fermenting reactor has a plurality of inlet and outlet openings through which starting material or fermented product can be selectively introduced and/or extracted.

11. A fermenter according to claim 10, comprising a central conveying station by which fermented product can be extracted through outlet openings and starting material or fermented product can be introduced as inoculum through inlet openings, wherein the flow paths to and from the openings can be reversed via the conveying station and respectively varying material flow profiles are formed in the fermenting reactor.

12. A fermenter according to claim 10, comprising an agitator the direction of rotation of which can be reversed during the fermenting process via a timing control or in response to parameters of fermentation.

13. A fermenter according to claim 11, wherein neighboring agitator blades overlap in axial direction.

14. A fermenter according to claim 11, wherein an agitator shaft of the agitator is supported on both sides and is dimensioned such that by the buoyancy it is centrally supported in the reactor.

15. A fermenter according to claim 10, wherein at the bottom of the fermenting reactor two impurities/settled material conveying means, are provided by which settled material is adapted to be conveyed toward a central settled material extract opening disposed between said pusher plates.

16. A fermenter according to claim 15, comprising a cleaning plant for washing the settled material.

17. A fermenter according to claim 10, wherein the fermenting reactor is arranged horizontally and has a circular or approximately rectangular cross-section, wherein in the latter case two inclined surfaces and a horizontal surface disposed therebetween form the bottom.

18. A fermenter according to claim 10, comprising a gas injection device for biogas.

19. A fermenter according to claim 18, wherein gas injection nozzles are arranged in the bottom of the fermenting reactor.

20. A fermenter according to claim 17, wherein the gas injection nozzles are arranged in the inclined surfaces.

21. A fermenter according to claim 20, wherein the gas injection nozzles open in vertical direction or normal to the inclined surfaces.

22. A fermenter according to claim 18, wherein the gas injection nozzles are combined into several fields which can be controlled independently of each other.

23. A fermenter according to claim 10, wherein the shell of the fermenting reactor is heated.

24. A fermenter according to claim 11, comprising a starting material direct charging through which starting material can be introduced after being heated independently of the conveying station.

25. A fermenter according to claim 10, wherein the fermenting reactor is composed of segments ready for transport.
